(19) United States
(12) Patent Application Publication
(10) Pub. No.: US 2017/0194667 A1
(43) Pub. Date: Jul. 6, 2017

(54) BATTERY AND BATTERY SYSTEM

(71) Applicant: OLYMPUS CORPORATION, Tokyo (JP)

(72) Inventors: Shoei TSURUTA, Tokyo (JP); Akira MATSUI, Tokyo (JP); Kaoru MATSUKI, Kawasaki-shi (JP)

(73) Assignee: OLYMPUS CORPORATION, Tokyo (JP)

(21) Appl. No.: 15/467,487

(22) Filed: Mar. 23, 2017

Related U.S. Application Data
(63) Continuation of application No. PCT/JP2014/080232, filed on Nov. 14, 2014.

(51) Int. Cl. H01M 10/42; H02J 7/00; H01M 2/10; A61B 17/28; H01M 10/0525; H01M 10/44

(52) U.S. Cl. CPC H01M 10/425; H01M 10/0525; H01M 10/44; H01M 2/1016; A61B 17/28; H02J 7/0045; A61B 2017/00734

(57) ABSTRACT

A battery (1) comprises a chargeable and dischargeable battery cell (23); a first electrode (21) and a second electrode (22) connected to the battery cell and electrically connected to an external electrode in a non-contact state; a switching circuit (24) which is provided in a battery circuit comprising the battery cell, the first electrode and the second electrode, and switches the current flowing in the battery circuit to alternating current or direct current; and an insulating housing (10) which houses the battery cell, the first electrode, the second electrode and the switching circuit therein.

The present invention relates to a battery and a battery system. This application is a continuation application based on a PCT International Application No. PCT/JP2014/080232, filed on Nov. 14, 2014. The content of the PCT International Application is incorporated herein by reference.

### FIELD OF THE INVENTION

### Description of Related Art

In recent years, medical devices have been becoming wireless, and types of treatment tools in which power is supplied from a battery have started to be proposed.

In that case, lithium ion batteries with high energy density per unit mass are expected to be generally utilized.

A general battery includes a battery cell capable of being charged and discharged, a conductive terminal for being electrically connected to an external charger or a medical device and the like (see, for example, Japanese Patent No. 4554222). When the battery is charging or discharging, a terminal of the battery and a conductive terminal or the like provided in the charger or the like are brought into contact with each other to electrically connect the terminals together.

### SUMMARY OF THE INVENTION

According to a first aspect of the present invention, a battery comprises: a chargeable and dischargeable battery cell; a first electrode and a second electrode connected to the battery cell and electrically connected to an external electrode in a non-contact state; a switching circuit which is provided in a battery circuit comprising the battery cell, the first electrode and the second electrode, and switches the current flowing in the battery circuit to an alternating current or a direct current; and an insulating housing which houses the battery cell, the first electrode, the second electrode and the switching circuit therein.

According to a second aspect of the present invention, there is provided a battery system comprising: the battery of the present invention; and a connecting device which has a recess in which the battery is loaded, and a connecting device side first electrode and a connecting device side second electrode disposed along inner surfaces of the recess therein, wherein when the battery is loaded in the recess, the first electrode and the second electrode face the connecting device side first electrode and the connecting device side second electrode to be capacitively coupled.

### BRIEF DESCRIPTION OF THE DRAWINGS

FIG. 1 is a perspective view showing a battery according to a first embodiment of the present invention.

FIG. 2 is a cross-sectional view of the battery.

FIG. 3 is a perspective view showing a battery system equipped with the battery and a charger.

FIG. 4 is a schematic partial sectional view of the charger.

FIG. 5 is a circuit diagram of charging.

FIG. 6 is a perspective view showing a treatment tool on which the battery is mounted.

FIG. 7 is a circuit diagram of discharging to the treatment tool.

FIG. 8 is a cross-sectional view showing an example of a state in which the battery is loaded on the treatment tool.

FIG. 9 is a cross-sectional view showing an example of a state in which the battery is loaded on the treatment tool.

FIG. 10 is a perspective view showing a battery according to a second embodiment of the present invention.

FIG. 11 is a cross-sectional view of the battery.

FIG. 12 is a perspective view showing a modified example of the battery.

FIG. 13 is a perspective view showing a modified example of the battery of the present invention.

FIG. 14 is a schematic cross-sectional view showing an example of electrode arrangement in the modified example.

FIG. 15 is a perspective view showing a modified example of the battery of the present invention.

### DETAILED DESCRIPTION OF THE INVENTION

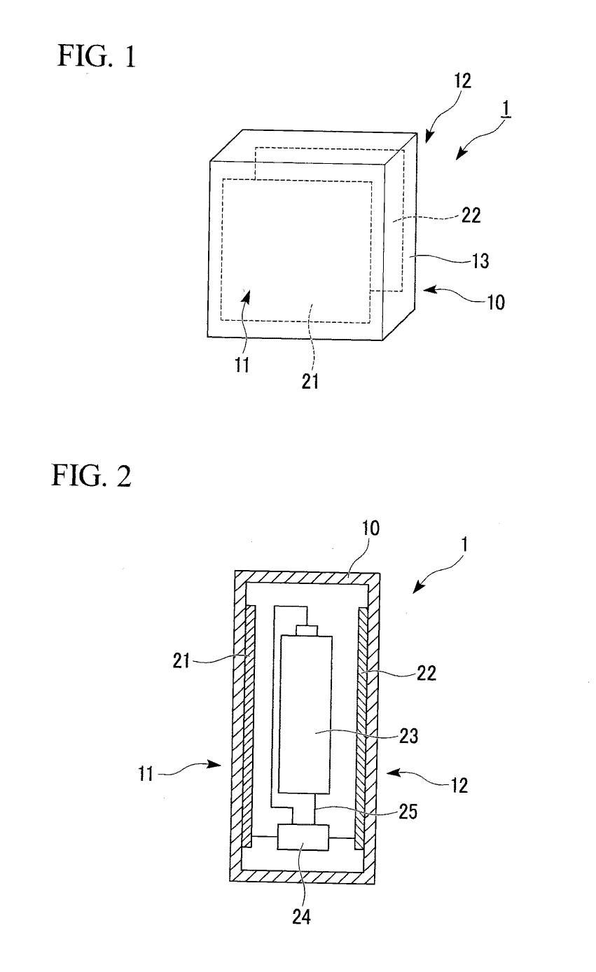

Hereinafter, a first embodiment of the present invention will be described with reference to FIGS. 1 to 9.

FIG. 1 is a perspective view showing a battery 1 of the present embodiment. The battery 1 comprises an insulating housing 10 which constitutes an outer surface of the battery 1, and a first electrode 21 and a second electrode 22 disposed inside the housing 10.

The housing 10 is formed of an insulating material. A resin is preferable as a material for forming the housing 10. For example, polycarbonate, a fluororesin, polyether ether ketone (PEEK) and the like can be used as the material. The dielectric constant of the insulating material forming the housing 10 is preferably 2 or more. When the housing 10 is made of a high dielectric constant material with a dielectric constant of 2 or more, it is possible to increase the electrostatic capacitance generated at the time of power transmission/reception to be described later, and to lower the voltage value applied to the electrode at the time of power transmission/reception.

FIG. 2 is a cross-sectional view of the battery 1, and shows a state seen from a right side surface 13 side shown in FIG. 1. A battery cell 23 capable of being charged and discharged, and a switching circuit 24 are housed inside the housing 10. The switching circuit 24 is electrically connected to the battery cell 23, a first electrode 21 and a second electrode 22. The first electrode 21, the second electrode 22, the battery cell 23 and the switching circuit 24 are connected by a wiring 25 to form a battery circuit.

The switching circuit 24 has two functions. One of the functions is to switch the current flowing inside the battery circuit between AC and DC, and the other thereof is to switch whether to discharge the AC current to the outside of the battery or to charge the battery cell with the DC current. Thus, the DC current flows through the battery cell 23 side of the switching circuit 24, the AC current flows through the first electrode 21 and the second electrode 22, and the discharging and charging modes are switched.

Even if the battery does not have a charging and discharging mode switching function, the battery can be used as, for example, a disposable battery that can only be discharged.

As the battery cell 23, any battery cell can be used as long as it can be charged and discharged, and for example, battery cells of various known structures such as a lithium ion battery cell can be appropriately selected and used.

The first electrode 21 and the second electrode 22 are formed in a planar shape by a conductor material and are symmetrically disposed to extend along a front surface 11 and a back surface 12 of the housing 10, respectively. As a material for forming the first electrode 21 and the second electrode 22, for example, a metal foil or the like can be used.

The switching circuit 24 is not particularly limited as long as it has a DC/AC conversion function, and a well-known converter circuit or the like can be appropriately selected in consideration of the size of the battery 1 and the like.

With the above-described configuration, the entire outer surface of the battery 1 is covered with the insulating housing 10 such that and the conductive member such as a terminal or an electrode is not exposed at all through the outer surface.

Next, the operation when the battery 1 is used will be described. The battery 1 can be used as a battery system in combination with a connecting device for transmitting and receiving power to and from the battery 1.

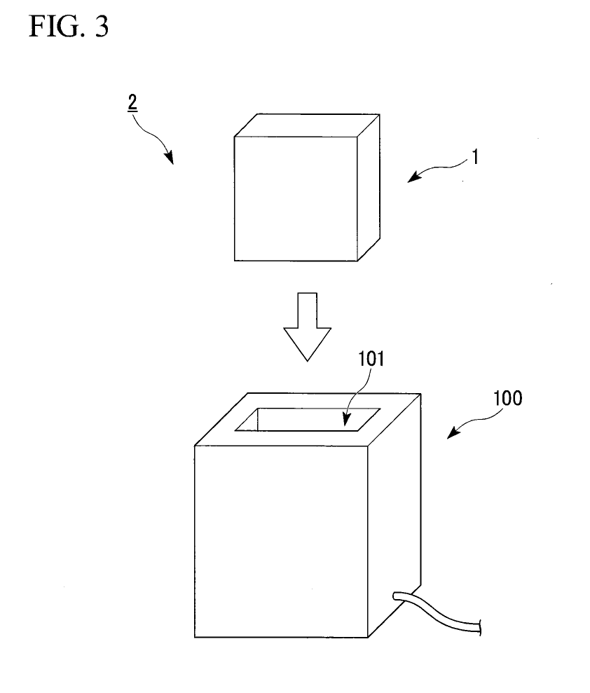

FIG. 3 shows a battery system 2 comprising a battery 1, and a charger (connecting device) 100 for charging the battery 1. The charger 100 has a recess 101 capable of housing the battery 1, and the entire outer surface of the charger 100 comprising the recess 101 is formed to be covered with an insulating material such as a resin.

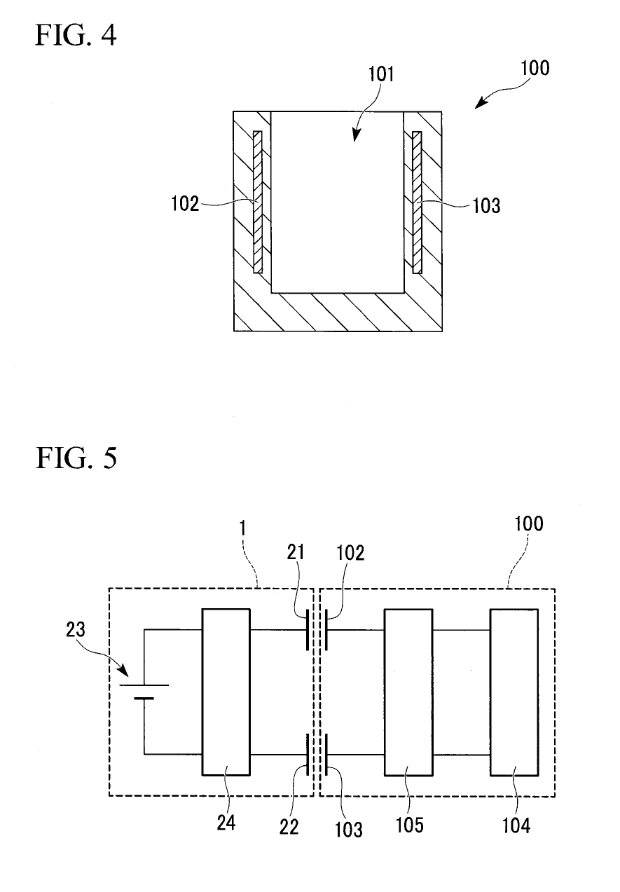

FIG. 4 is a diagram schematically showing a cross section of the charger 100. The charger 100 comprises a planar first power transmission electrode (a connecting device side first electrode) 102 and a second power transmission electrode (a connecting device side second electrode) 103. The first power transmission electrode 102 and the second power transmission electrode 103 are disposed along the two facing surfaces among the inner surfaces of the recess 101 so as not to be exposed.

To charge the battery 1, a user loads the battery 1 into the recess 101 such that two surfaces on which the first power transmission electrode 102 and the second power transmission electrode 103 are disposed face the front surface 11 and the back surface 12 on which the first electrode 21 and the second electrode 22 are disposed.

FIG. 5 is a circuit diagram showing a state in which the battery 1 is loaded in the recess 101 as described above. Since the first power transmission electrode 102 and the second power transmission electrode 103 face the first electrode 21 and the second electrode 22, the facing electrodes are capacitively coupled (electric field coupling) in a non-contact state to form a circuit which comprises the battery 1 and the charger 100. The thickness of the housing 10 is set in advance to enable the above-described capacitive coupling. In FIG. 5, reference numeral 104 denotes a power supply circuit, and reference numeral 105 denotes a power transmission circuit for adjusting the mode of a current which is transmitted from the charger 100 to the battery 1.

When a high-frequency AC current is supplied from the charger 100 in the state in which the above-described circuit is formed, power can be transmitted to the battery 1 via the capacitively coupled electrodes. By converting the AC current transmitted from the charger 100 into a DC current by the switching circuit 24, the battery cell 23 can be charged.

Since the AC current is supplied from the charger 100, as long as the first power transmission electrode 102 and the second power transmission electrode 103 face the first electrode 21 and the second electrode 22, a correspondence relation of individual electrodes is not a problem, and charging can be performed in any correspondence relation. That is, the first electrode 21 may be disposed to face the first power transmission electrode 102, or may be disposed to face the second power transmission electrode 103.

As shown in FIG. 1 or the like, the housing 10 of the battery 1 is formed in rectangular parallelepiped shape in which the front surface 11 and the back surface 12 are formed in a square shape. Since the front surface 11 and the back surface 12 are figures having rotational symmetry, the shape of the battery 1 is the same in a posture in which any one of the four surfaces other than the front surface 11 and the back surface 12 faces upward. Further, when any one of front surface 11 or back surface 12 is on the front side, its shape does not change. Therefore, when the battery 1 is loaded in the recess 101, the first power transmission electrode 102 and the second power transmission electrode 103 necessarily face the first electrode 21 and the second electrode 22 irrespective of the direction thereof, and charging can be performed.

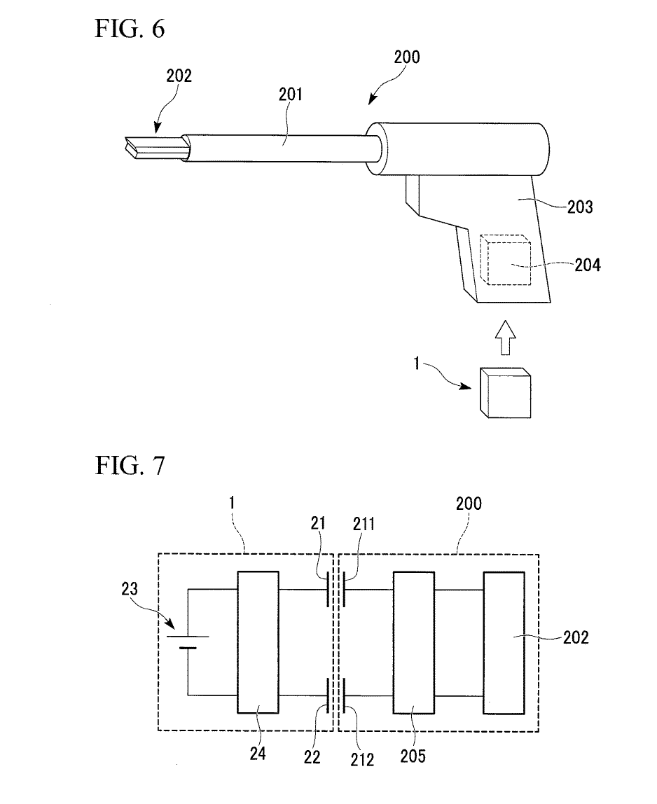

After the battery 1 is charged, the battery is mounted on a predetermined discharging device (connecting device) and used as a power supply. FIG. 6 shows a grasping forceps 200 which is a treatment tool comprising a rigid insertion unit 201 and a treatment unit 202 provided at a distal end portion of the insertion unit 201, as an example of a discharging device. The target discharging device is not limited to a treatment tool, and it can be applied without particular limitation as long as it is used by being energized.

A handle 203 of the grasping forceps 200 is provided with a recess 204 for housing the battery 1. The shape of the recess 204 may be the same as that of the recess 101 of the charger 100. The grasping forceps 200 comprises a pair of electrodes for receiving power of the first power reception electrode (the connecting device side first electrode) and the second power reception electrode (the connecting device side second electrode). Although it is not shown in FIG. 6, the first power reception electrode and the second power reception electrode are disposed along the two facing surfaces among the inner surfaces of the recess 204 so as not to be exposed to the outside. That is, the pair of power receiving electrodes are housed inside the grasping forceps 200.

FIG. 7 is a circuit diagram of a circuit formed when discharging from the battery 1 to the grasping forceps 200 is performed. Like charging, when the first power reception electrode 211 and the second power reception electrode 212 face the first electrode 21 and the second electrode 22, the facing electrodes are capacitively coupled with each other. When discharging from the battery 1 is performed, the DC current extracted from the battery cell 23 is converted into an AC current by the switching circuit 24 and is transmitted to the grasping forceps 200. In the grasping forceps 200, the AC current supplied from the battery 1 is appropriately adjusted by the power reception circuit 205 and is supplied to the treatment unit 202 which is a load.

Like charging, when the battery 1 is loaded in the recess 204, it is possible to perform discharging to the grasping forceps 200 irrespective of the direction.

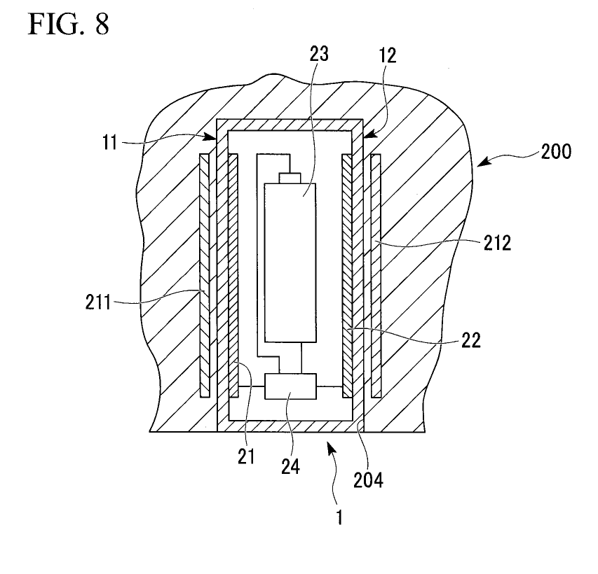

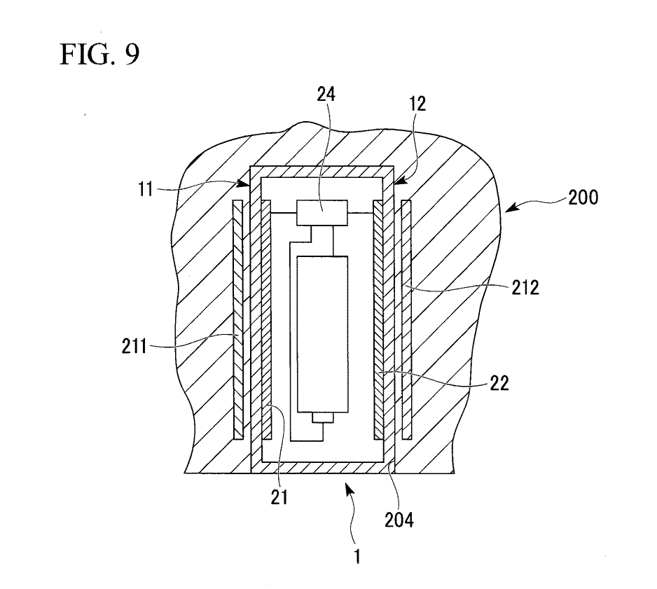

That is, as shown in FIG. 8, the battery 1 may be loaded in the recess 204 in the same posture as shown in FIG. 2, or as shown in FIG. 9, the battery 1 may be loaded in the recess 204 in a posture vertically reversed from the posture of FIG. 8. Further, even when the battery 1 is loaded in the recess 204 in the posture in which the front surface 11 and the back surface 12 are reversed from the posture shown in FIGS. 8 and 9, it is possible to perform discharging from the battery 1 to the grasping forceps 200.

When a high-frequency current is used in the discharging device to be applied, the supplied AC current may be used as it is by adjusting the voltage or the current value or the like by the power reception circuit 205. When the DC current is used in the discharging device, a converter circuit or the like may be appropriately provided in the power reception circuit 205 so that the supplied AC current can be converted into the DC current.

As described above, the battery 1 according to the present embodiment can be electrically connected to a connecting device such as a charger or a medical device, without using a conductive terminal such as a metal. Therefore, it is possible to receive and transmit power from and to the connecting device, while providing a configuration in which the entire outer surface is covered with the insulating housing 10, and it can be suitably used as a battery. Unlike a normal battery, both of the input to the battery 1 and the output from the battery 1 are AC. The frequency of the AC is preferably the frequency of a high frequency band of about 100 kHz to 1 GHz.

Further, since the battery 1 has no portion which is formed of a conductor such as a terminal that is exposed through the outer surface and is connected with the internal mechanism by a conductor such as wiring, for example, there is no need to take care to prevent the terminal from becoming wet and it is easy to handle.

Furthermore, since there is no need to bring the connecting device into contact with terminals and the like in order to transmit and receive power, a freedom of loading to the connecting device can be set to a higher degree.

In the present invention, in a battery system comprising a battery and at least one connecting device, among the postures in which the battery can be loaded in the recess of the connecting device, a posture in which the first electrode and the second electrode of the loaded battery face the connecting device side first electrode and the connecting device side second electrode provided in the connecting device to enable the capacitive coupling is defined as a ‘power transmittable and receivable posture.’ As described above, in the battery system 2, all postures in which the battery can be loaded in the recess of the connecting device are power transmittable and receivable postures, and there are a total of eight power transmittable and receivable postures.

Since an ordinary battery cannot perform the power transmission and reception unless the connecting device is brought into contact with the terminal or the like, there is basically only one posture in which power can be transmitted to and received from a single connecting device. In the battery system of the present invention, by suitably changing the external shape of the battery 1 substantially determined by the shape of the housing 10, the shape of the recess of the connecting device, the arrangement of the first electrode 21 and the second electrode 22, and the arrangement of the electrodes of the connecting device, it is possible to set the power transmittable and receivable postures to an arbitrary number of 2 or more.

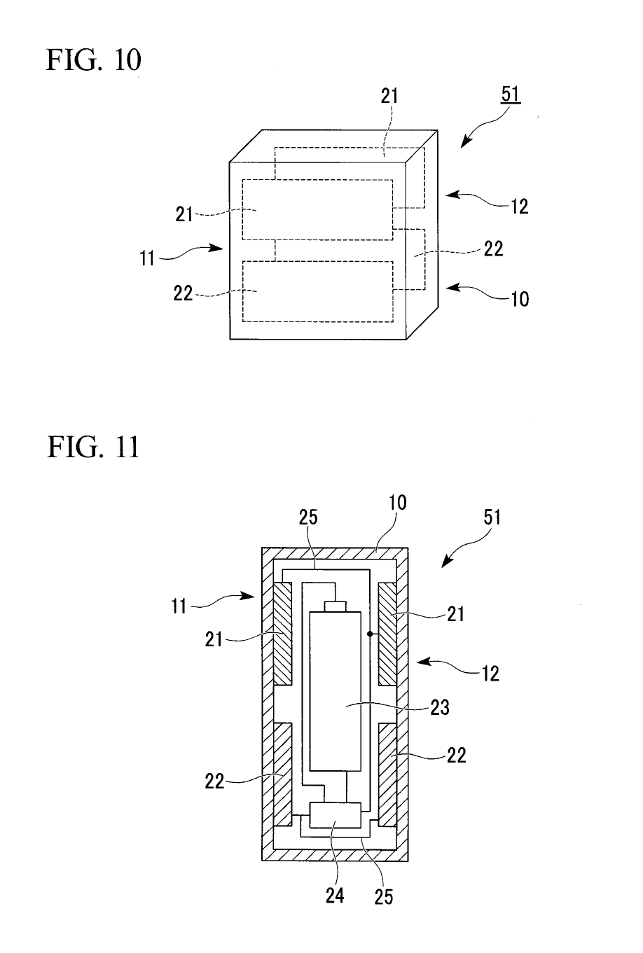

Next, a second embodiment of the present invention will be described with reference to FIGS. 10 and 11. A battery 51 of the present embodiment is different from the aforementioned battery 1 in the manner in which the first electrode and the second electrode are arranged. In the following description, the same configurations as those already described are denoted by the same reference numerals, and a repeated explanation will not be provided.

As shown in FIG. 10, in the battery 51, first electrodes 21 and second electrodes 22 are disposed on each of the front surface 11 and the back surface 12. That is, the battery 51 comprises two first electrodes 21 and two second electrodes 22.

FIG. 11 is a cross-sectional view of the battery 51. The two first electrodes 21 and the two second electrodes 22 are connected by wiring 25 and are at the same potential (same voltage and same phase).

Although it is not shown, in the connecting device connected to the battery 51, a connecting device side first electrode and a connecting device side second electrode are also disposed along each of two surfaces facing the front surface 11 and the back surface 12 among the inner surfaces of the recess when the battery 51 is housed.

Similarly to the battery 1 of the first embodiment, the battery 51 of the present embodiment is very easy to handle and can constitute a battery system having a high freedom of loading into the connecting device.

Further, since the first electrodes 21 and the second electrodes 22 are provided on each of the front surface 11 and the back surface 12, if the battery 51 moves in the recess in the front-rear direction (the direction between the front surface 11 and the back surface 12) while loaded on the connecting device, the distance between the facing electrodes becomes longer in one of the front surface 11 and the back surface 12, but the distance between the facing electrodes becomes shorter in the other thereof. Therefore, the combined capacitance of the capacitor established between the battery 51 and the connecting device is hard to change, and in the circuit formed by the battery 51 and the connecting device, the capacitance stability of the capacitor is remarkably improved and the control is easy. As a result, it is possible to perform more stable charging and discharging.

However, depending on the posture in which the battery 51 is loaded on the connecting device, the manner in which the connecting device side first electrode and the connecting device side second electrode are arranged and the like, the first electrodes 21 and the second electrodes 22 may face both of the connecting device side first electrode and the connecting device side second electrode. Because the power transmission and reception are not performed in such a posture, it should be noted that there are cases in which the number of power transmittable and receivable postures becomes less than that of the battery 1.

In the present embodiment, a first electrode 21 and a second electrode 22 may be provided on only one of the front surface and the back surface of the battery. In contrast, in the connecting device, the connecting device side first electrode and the connecting device side second electrode may be disposed only in one of the two surfaces facing the front surface 11 and the back surface 12 when the battery 51 is housed.

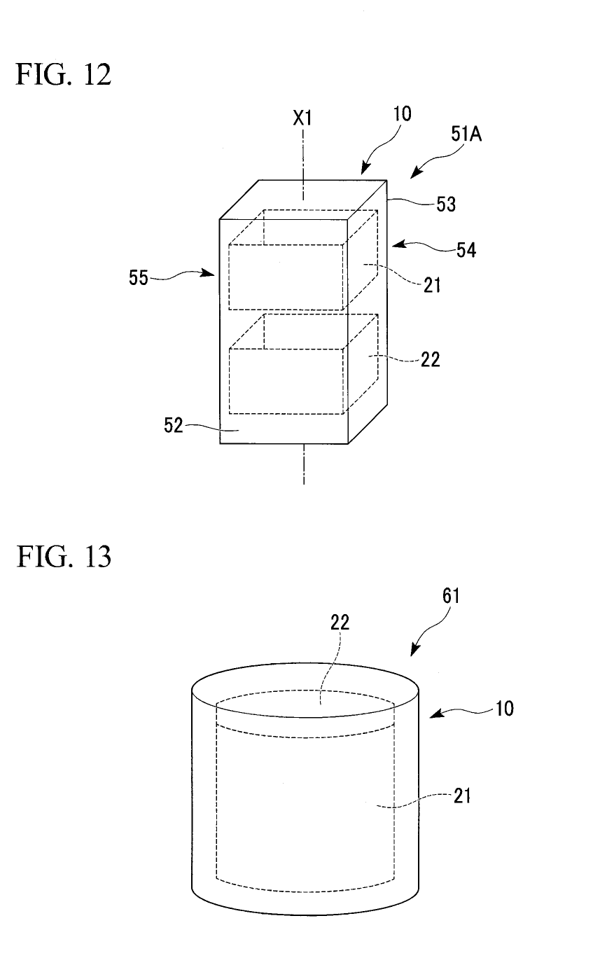

When the first electrodes 21 and the second electrodes 22 are provided on a plurality of surfaces, the first electrodes or the second electrodes on each surface may be connected to each other. A battery 51A of the modified example shown in FIG. 12 has a square columnar external shape with a square bottom, and the first electrodes 21 and the second electrodes 22 are disposed along each of the four outer surfaces 52, 53, 54 and 55. Since the first electrodes 21 and the second electrodes 22 on each outer surface are connected to each other, the battery 51A has a structure in which the first electrode 21 and the second electrode 22 are disposed on the outer surface over the entire perimeter. Since the battery 51A has such a structure, even if the battery 51A is loaded without considering the relative positional relations with the connecting device in the direction around an axis X1 of the square column when loaded in the recess, it can transmit or receive power to and from the connecting device.

Further, if the external shape of the battery 51A is formed in a columnar shape with a circular bottom surface, when housed in the recess of the connecting device, even if the battery 51A is housed in the recess without considering the positional relation with the connecting device in the circumferential direction (the direction around the columnar axis), it is possible to transmit and receive power. In this case, there are innumerable power transmittable and receivable postures in the battery system.

Although the respective embodiments of the present invention have been described above, the technical scope of the present invention is not limited to the above-described embodiments, and it is possible to change combinations of constituent elements or to add or delete various changes to the respective constituent elements within a scope that does not depart from the spirit of the present invention.

First, the external shape of the battery is not limited to the aforementioned shape, and the battery may be formed in any way as long as the battery can be housed in the recess of the connecting device and has two or more power transmittable and receivable postures.

For example, as the battery to be loaded in the charger 100 shown in FIG. 3, there is no restriction on the shape of the front and back surfaces of the housing as long as the battery can be loaded in the recess 101, and the shapes and sizes of the front surface and the back surface may be different. Also, the shapes of the surfaces to be capacitively coupled may be different between the battery and the connecting device.

Further, the shapes and sizes may be different between the electrode of the battery side and the electrode on the connecting device side which are capacitively coupled with each other.

Furthermore, the external shape of the battery and the shape of the recess do not need to be exactly the same. Therefore, when the battery is loaded in the recess, even if a part of the battery protrudes from the recess or a space remains in the recess, as long as the electrode on the battery side and the electrode on the connecting device side face each other so that the electrodes can be capacitively coupled with each other, they function as a battery system without problems.

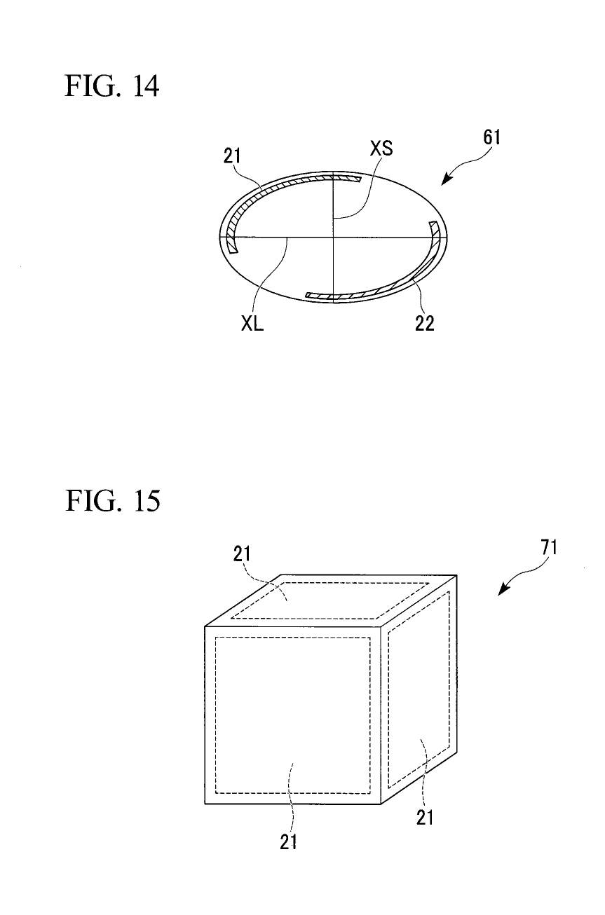

Furthermore, the external shape of the battery is not limited to a shape in which the outer surface includes only a flat surface. Accordingly, the external shape may have the above-described columnar shape or an elliptical columnar shape in which both sides are elliptical in the axial direction or a polygonal columnar shape with rounded corners or ridges, such as the battery 61 shown in FIG. 13. At this time, it is not necessary to arrange the first electrode and the second electrode in parallel with a major axis or a minor axis of the elliptical surface on both sides of the elliptical column in the axial direction. For example, as schematically shown in FIG. 14, the first electrode and the second electrode may be disposed to face each other in a direction which is parallel with neither the minor axis XS nor the major axis XL.

In the modified example shown in FIG. 15, the external shape of the battery 71 is a cube, and the first electrodes 21 are disposed on each of three mutually adjacent surfaces shown in FIG. 15. The second electrodes 22 are disposed on each of the remaining three surfaces which are not shown in FIG. 15, and the battery 71 has three first electrodes 21 and three second electrodes 22.

As the connecting device of the battery 71, a device that has a cubic recess and has the connecting device side first electrode and the connecting device side second electrode disposed on a pair of facing surfaces among the recess inner surfaces is prepared. In the battery system having such a configuration, when the battery 71 is loaded in the recess, regardless of the loading postures of the battery 71, one of the three first electrodes 21 necessarily faces one of the connecting device side first electrode and the connecting device side second electrode, and one of the three second electrodes 22 faces the other of the connecting device side first electrode and the connecting device side second electrode. Therefore, in the battery system, there are twenty-four power transmittable and receivable postures, and usability can be remarkably improved.

Further, in each of the above-described embodiments, switching between charging and discharging using the switching circuit may be performed automatically, for example, by identifying a device to which the battery is connected, or may be configured such that a user designates the switching mode. In the latter case, a switch for switching the mode may be provided on the outer surface of the battery.

What is claimed is:

1. A battery comprising:
a chargeable and dischargeable battery cell;
a first electrode and a second electrode connected to the battery cell and configured to be electrically connected to an external electrode in a non-contact state;
a switching circuit which is provided in a battery circuit comprising the battery cell, the first electrode and the second electrode, and switches the current flowing in the battery circuit to an alternating current or a direct current; and
an insulating housing which houses the battery cell, the first electrode, the second electrode and the switching circuit therein.

2. The battery according to claim 1, wherein the first electrode and the second electrode are disposed symmetrically with respect to a predetermined axis set in the housing.

3. The battery according to claim 1, wherein the switching circuit is configured to switch between a charging mode and a discharging mode.

4. A battery system comprising:
the battery according to any one of claim 1; and
a connecting device which has a recess in which the battery is loaded, and a connecting device side first electrode and a connecting device side second electrode disposed along inner surfaces of the recess therein,
wherein, when the battery is loaded in the recess, the first electrode and the second electrode face the connecting device side first electrode and the connecting device side second electrode to be capacitively coupled.

5. The battery system according to claim 4, wherein the battery can be loaded in the recess in a plurality of postures, and the battery has two or more power transmittable and receivable postures in which the first electrode and the second electrode face the connecting device side first electrode and the connecting device side second electrode to be capacitively coupled when the battery is loaded in the recess.

6. The battery system according to claim 5, wherein the battery has two or more first electrodes, and in the power transmittable and receivable postures, at least one of the first electrodes faces one of the connecting device side first electrode and the connecting device side second electrode to be capacitively coupled.
